(19) United States
(12) Patent Application Publication
(10) Pub. No.: US 2023/0141973 A1
(43) Pub. Date: May 11, 2023

(54) OZONE GENERATOR FOR OZONE SANITIZATION AND DISINFECTION OF ICE MAKERS

(71) Applicant: YANTAI UNITED OZONETEC CORPORATION, Yantai (CN)

(72) Inventors: MIing-Yung Hsu, Yantai (CN); Wei-Chun Hsu, Yantai (CN)

(21) Appl. No.: 17/519,571

(22) Filed: Nov. 5, 2021

(51) Int. Cl. F25C 1/00; A61L 2/20

(52) U.S. Cl. CPC F25C 1/00; A61L 2/202; A61L 2202/11; A61L 2202/14; A61L 2202/15; F25C 2400/12; F25C 2700/14

(57) ABSTRACT

An ozone generator for producing ozone water for the sanitation and disinfection of an ice maker includes cathode plates and anode plates that are arranged alternatively in an electrolysis chamber. The cathode plates and the anode plates are connected to a cathode wire and an anode wire of a cable. A wiring chamber is provided with a temperature sensor extending into the electrolysis chamber and connected with a temperature sensor wire of the cable. The cable is connected to a control circuit. The ozone generator is submerged in the ice-making water of the ice-making water tank. The temperature sensor of the ozone generator submerged in the ice-making water is configured to sense temperature changes. When the ice maker is activated, the temperature of the ice-making water in the ice-making water tank will drop. When the temperature drops to the set temperature, the ozone generator is activated once to produce ozone gas. The ozone gas is dissolved in the ice-making water to generate ozone water. The ozone water is used to sanitize the ice-making loop process of the ice maker. The ozone generator has simple structure, low cost and good effect.

### FIELD OF THE INVENTION

The present invention relates to an ozone generator, and more particularly to an ozone generator for ozone sanitization and disinfection of an ice maker.

### BACKGROUND OF THE INVENTION

An ice maker is equipped with an ice-making water tank. The tap water in the ice-making water tank is susceptible to contamination and growth of microorganisms, such as bacteria, yeasts, fungi, and molds. The tap water is pre-treated with a filtering process to remove residual chlorine which then flows into the ice-making water tank, which is easy to contaminate with and further breed bacteria and molds. As a result, bacteria in the ice cubes produced by the ice maker may seriously exceed acceptable health standard. Some well known fast-food restaurants provide ice cubes with excessive bacteria these incidents are often reported by the media. This not only may seriously affect people’s health but also bring my bring unwanted liability and negative attention to the business activities of the food and beverage industry. Ozone can be used in this field as an efficient, broad-spectrum, and residue-free sanitization technology. Some ice makers on the market use ozone for sanitation, and some ice makers use high-pressure discharge method to ionize air to generate ozone for sanitization and disinfection. Because of its inevitable production of nitrogen oxides that are carcinogens, they do not meet the requirements of hygiene and health and are difficult to be widely used. Prior art discloses a device for sterilizing an ice machine using ozone gas from an electrolytic ozone generator. It describes an ozone generator that produces ozone by electrolyzing pure water. When the ozone generator by electrolyzing pure water works, it produces ozone gas, and then mixes the ozone gas with tap water to produce ozone water. The ozone gas and ozone water are configured to disinfect and sanitize the ice machine. No nitrogen oxides are produced, and the structure is simplified. Because the ozone generator needs to use high-purity water and cannot directly electrolyze tap water, the structure is still relatively complicated and has high costs associated. These pure water generators have a difficult time achieving economic benefits for the user, so they are not widely used.

### SUMMARY OF THE INVENTION

The primary object of the present invention is to overcome the above-mentioned shortcomings of the prior art and to provide an ozone generator for ozone sanitization and disinfection of an ice maker, which is suitable for various types of ice makers.

The technical solution provided by the present invention is described below. An ozone generator for ozone sanitization and disinfection of an ice maker comprises a generator housing. The generator housing has a wiring chamber and an electrolysis chamber therein. Cathode plates and anode plates are provided in the electrolysis chamber. The cathode plates and the anode plates are arranged alternatively. The cathode plates and the anode plates are connected to a cathode wire and an anode wire of a cable through a cathode conductive stud and an anode conductive stud passing through the wiring chamber, respectively. The wiring chamber is provided with a temperature sensor extending into the electrolysis chamber. The temperature sensor is connected with a temperature sensor wire of the cable. The cable is connected to a control circuit.

Preferably, each of the anode plates is a coated titanium anode, and each of the cathode plates is a titanium cathode or stainless steel cathode. The coated titanium anode includes a titanium substrate and a doped tin dioxide coating.

Preferably, the cable extends out of the generator housing through a gasket, and the wiring chamber is filled with epoxy resin sealant.

Preferably, the ozone generator is submerged in ice-making water of an ice-maker’s water tank. The temperature sensor of the ozone generator submerged in the ice-making water is configured to sense temperature changes. When the ice maker is activated, the temperature of the ice-making water in the ice-making water tank drops. When the temperature drops to a set temperature, the ozone generator is activated once to produce ozone gas. The ozone gas is dissolved in the ice-making water to generate ozone water. The ozone water is used to sterilize an ice-making loop process of the ice maker.

Preferably, the set temperature is between 4-20° C. With the optimal set temperature at 5° C.

The beneficial effects of the present invention are described below.

The present invention adopts an ozone generator for electrolyzing ice-making water, which is directly submerged into an ice-making water tank. The ozone generator for electrolyzing ice-making water has an extremely simple structure and low cost. The ozone gas produced by the ozone generator for electrolyzing ice-making water does not contain harmful gases such as nitrogen oxides. The ozone generator for electrolyzing ice-making water is directly installed in the ice-making water tank. When the temperature sensor senses that the temperature of the ice-making water drops, it is confirmed that the ice maker is started for making ice cubes. When the temperature drops to the set temperature, the control circuit controls the ozone generator to work. When it is working, ozone gas microbubbles are generated. At the moment when ozone gas microbubbles are generated, the ozone gas microbubbles are quickly dissolved and mixed in the ice-making water to directly generate ozone water. Ozone water can sterilize the water in the ice-making water tank and the pipeline through which the ozone water flows to the spray nozzle and the ice mold cavity. In addition, a small amount of ozone gas of the ozone water is dispersed into the air, so that the area around the ice-making water tank forms an ozone gas environment, which has a bacteriostatic effect inside the ice maker. The ozone water in the ice-making water tank is disinfected to become sanitized ice-making water. The ice-making water finally flows through the ice mold cavity to make ice cubes without bacteria. In effect, a new or recently sanitized ice-maker will self maintain a sanitary environment in the ice- maker to consistently and automatically deliver sanitized ice.

### BRIEF DESCRIPTION OF THE DRAWINGS

FIG. 1 is a structural schematic view of the present invention.

### DETAILED DESCRIPTION OF THE PREFERRED EMBODIMENTS

Embodiments of the present invention will now be described, by way of example only, with reference to the accompanying drawings.

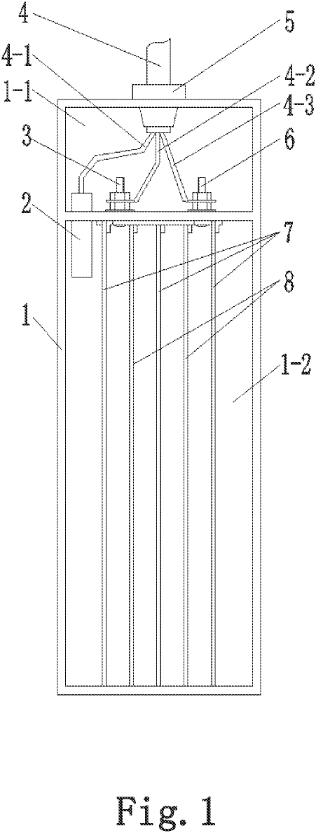

As shown in FIG. 1, an ozone generator for ozone sanitization and disinfection of an ice maker comprises a generator housing (1). The generator housing (1) has a wiring chamber (1-1) and an electrolysis chamber (1-2) therein. Cathode plates (7) and anode plates (8) are provided in the electrolysis chamber (1-2). Each anode plate (8) is a coated titanium anode. Each cathode plate (7) is a titanium cathode or stainless steel cathode. The cathode plates (7) and the anode plates (8) are arranged alternatively. The cathode plates (7) and the anode plates (8) are connected to a cathode wire (4-3) and an anode wire (4-2) of a cable (4) through a cathode conductive stud (6) and an anode conductive stud (3) passing through the wiring chamber (1-1), respectively. The wiring chamber (1-1) is provided with a temperature sensor (2) extending into the electrolysis chamber (1-2). The temperature sensor (2) is connected with a temperature sensor wire (4-1) of the cable (4). The cable (4) is connected to a control circuit.

The cable (4) composed of the cathode wire (4-3), the anode wire (4-2) and the temperature sensor wire (4-1) extends out of the generator housing (1) through a gasket (5). The wiring chamber (1-1) is filled with epoxy resin sealant, which can avoid the oxidative corrosion and electric corrosion of the exposed conductive wires immersed in the ozone water in the water.

When the ice maker is started for making ice cubs, tap water flows into the ice-making water tank. The ice maker is pre-cooled, so that the water temperature of the ice-making water tank continues to drop. The temperature sensor (2) transmits the temperature signal to the control circuit through the temperature sensor wire (4-1) of the cable (4). When the temperature drops to the set temperature (preferably, 5° C.), the control circuit supplies constant current power supply to the anode plates and the cathode plates of the ozone generator through the cathode wire (4-3) and the anode wire (4-2) of the cable (4). The voltage range of the power supply is 3.5 to 16 V. Tap water is electrolyzed under the action of an electric field. Oxygen ions generate ozone gas microbubbles under the action of the anode catalyst. The ozone gas microbubbles are quickly dissolved and mixed into the tap water to directly generate ozone water to be exchanged with the water in the ice-making water tank to form ozone water, so that the ozone water is distributed throughout the ice-making water tank. When the proper concentration of ozone water is achieved, the control circuit stops power supply. The ozone water in the ice-making water tank is poured onto the ice mold cavity through a spray nozzle to gradually freeze, and it also has a sanitization and disinfection effect on the part where the ozone water flows. During this process, a small amount of ozone gas is dispersed into the air, so that the area around the ice-making water tank forms an ozone gas environment, which has a bacteriostatic effect inside the ice maker.

The control circuit can supply power regularly and change the current value to control the amount of ozone gas generated to produce the appropriate concentration of ozone water for sanitation. This control circuit allows the device to be suitable for various ice-makers of different specifications and models to achieve the effect of sanitization and disinfection. There are many ways to implement the circuit on the market, so it won’t be described hereinafter.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. An ozone generator for ozone sanitization and disinfection of an ice maker, comprising a generator housing (1), the generator housing (1) having a wiring chamber (1-1) and an electrolysis chamber (1-2) therein, cathode plates (7) and anode plates (8) being provided in the electrolysis chamber (1-2), the cathode plates (7) and the anode plates (8) being arranged alternatively, the cathode plates (7) and the anode plates (8) being connected to a cathode wire (4-3) and an anode wire (4-2) of a cable (4) through a cathode conductive stud (6) and an anode conductive stud (3) passing through the wiring chamber (1-1) respectively, the wiring chamber (1-1) being provided with a temperature sensor (2) extending into the electrolysis chamber (1-2), the temperature sensor (2) being connected with a temperature sensor wire (4-1) of the cable (4), the cable (4) being connected to a control circuit.

2. The ozone generator as claimed in claim 1, wherein each of the anode plates (8) is a coated titanium anode, each of the cathode plates (7) is a titanium cathode or stainless steel cathode; the coated titanium anode includes a titanium substrate and a doped tin dioxide coating.

3. The ozone generator as claimed in claim 1, wherein the cable (4) extends out of the generator housing (1) through a gasket (5), and the wiring chamber (1-1) is filled with epoxy resin sealant.

4. The ozone generator as claimed in claim 1, wherein the ozone generator is submerged in ice-making water of an ice-making water tank, the temperature sensor of the ozone generator submerged in the ice-making water is configured to sense temperature changes, when the ice maker is activated, the temperature of the ice-making water in the ice-making water tank drops, when the temperature drops to a set temperature, the ozone generator is activated once to produce ozone gas, the ozone gas is dissolved in the ice-making water to generate ozone water, the ozone water is used to sterilize an ice-making loop process of the ice maker.

5. The ozone generator as claimed in claim 4, wherein the set temperature is 4-20° C.

6. The ozone generator as claimed in claim 4, wherein the set temperature is 5° C.
